(19) United States
(12) Patent Application Publication
(10) Pub. No.: US 2002/0028232 A1
(43) Pub. Date: Mar. 7, 2002

(54) Wound dressing

(76) Inventor: Sunao Kubota, Tokyo (JP)

Correspondence Address: WENDEROTH, LIND & PONACK, L.L.P., 2033 K STREET N. W., SUITE 800, WASHINGTON, DC, 20006-1021, US

(21) Appl. No.: 09/852,695

(22) Filed: May 11, 2001

Related U.S. Application Data
(63) Continuation of application No. 08/490,996, filed on Jun. 15, 1995.

(51) Int. Cl. A61K 9/70

(52) U.S. Cl. 424/445; 442/123

(57) ABSTRACT

The present invention provides a wound dressing containing macromolecular compounds, wherein said macromolecular compounds comprise multiple blocks having a cloud point and hydrophilic blocks, have a sol-gel transition temperature in aqueous solutions, and indicate a liquid state reversibly at temperatures lower than said sol-gel transition temperature.

### FIELD OF THE INVENTION

The present invention relates to a material which is suitable for dressing a wound such as a burn. More particularly, the present invention relates to a wound dressing which can be removed easily from the wound when the dressing is to be replaced or the wound is healed, without substantially giving damage to the wound and without giving pain to the patient.

Furthermore, the present invention relates to a wound dressing that prevents infection and promotes healing of the wound.

### PRIOR ART AND PROBLEMS

Conventional wound dressings for dressing and protecting a wound such as a burn can be classified into the following two types by structure:

1) Materials represented by woven cloths such as a gauze, non-woven cloths and sponges whose structural feature is the presence of many apertures.

2) Film made of bio-derived materials such as chitin. collagen and fibrin, or of artificial materials such as polyurethane, silicone rubber and silicone gel, whose structural feature is extreme scarcity of apertures.

These materials have a different construction with its merits and demerits. The merits of the first type of wound dressing made of porous materials such as woven cloth, non-woven cloth, sponge, etc. include capability to retain a large quantity of exudate secreted from the wound and discharge it externally. These dressings thus prevent storage of exudate in the wound and decrease the frequency of infection, etc. Another merit of this type of wound dressing is that it is possible to apply antibacterial agents, anodynes, healing promoters or other medicines externally from outside the wound dressing, allowing the medicines to reach the wound through the dressing made of porous materials. Furthermore, the porous materials are flexible, and fit into a wound of a complicated form. In addition, oxygen, which is required for the wound, passes through the dressing freely.

The most serious problem of porous materials is that the exudate and the tissue enter the pores, which makes it extremely difficult to replace the dressing or remove it after healing. When one tries to remove the material forcefully, the healed tissues are damaged to delay the healing significantly. Further, it takes much time to remove the dressing and the patient suffers severe pain.

Water evaporation rate from the porous materials is very high so that the wound is excessively dry and the healing is delayed. Another problem, which is pointed out, is that because of the large number of apertures, external bacteria would easily get through the material and reach the wound.

The merits of the film-type materials with few apertures, on the other hand, include the fact that the difficulty of peeling off of the wound dressing from the wound, which is the most serious problem of the above-mentioned porous materials, is remarkably improved, that said materials themselves are a high barrier to bacteria, and that they limit evaporation of water from the wound to prevent the wound from drying.

The most serious problem of the materials with a small number of apertures is poor exudate absorbing capability, as compared with the materials of a high porosity. A large amount of exudate may be stored between the dressing and the wound providing a good opportunity for the bacteria to grow therein. Further, it is difficult to let the healing medicines get through the material to reach the wound. Various other problems remain unsolved with regard to the film-type materials such as difficulty to fit into the wound of a complex form.

Efforts have been made to solve the above problems. For example, to alleviate the difficulty of removing the material from the wound, one of the critical problems of the porous materials, porous rayon woven clothes are coated with vaseline ointments (S. Kawakami et al, “Clinical Effects of Non-Adherent Gauze (Adaptic) on Skin Deficiency” Clinical Report, 22, 1113, 1988). Another example is silicone-coated nylon woven clothes (R. Fujimori, “To Get Better Wound Dressing,” Rinsho Derma, 32, 1403, 1990). These materials reveal some improvements in terms of peelability, but peeling is very difficult when they are used for a wound with a deep deficiency zone for a long time.

To improve excessive storage of exudate from the wound, which is one of the critical problems of the film type materials with poor porosity, film-like materials are provided with slits, or water-absorbing non-woven cloths or hydrogels are laminated on the wound side of a film layer (M. Takamura et al, “Development of Antibacterial Agent-Containing Wound Dressing Comprising Polyurethane Film and Water-Absorbing Non-Woven Cloths—I, Basic Evaluation,” journal of Japan Society of Plastic and Reconstructive surgery, 12, 443, 1992, Fowler E. F. et al., A New Hydrogel Wound Dressing for the Treatment of Open Wounds, Ostomy/Wound Management, 37, 39, 1991). However, the water absorbing capability of the water-absorbing non-woven cloths and hydrogels is limited and some exudate is still stored on the wound compared with the porous materials if the volume of the exudate from the wound is large.

As described above, no effective wound dressings so far have the merits of both materials: a good exudate absorbing and discharging property, the property that various medicines externally applied on the dressing will reach the wound through the material (these are the merits of porous materials), and good peelability (the merit of non-porous material).

### SUMMARY OF THE INVENTION

The object of the present invention is to provide a new wound dressing for overcoming the above-stated problems of the conventional wound dressings of the prior art, namely, poor absorption of exudate secreted the wound and poor peelability frog the wound.

More specifically, the present invention provides a wound dressing containing macromolecular compounds that have a totally different property than conventional gels. More specifically, said macromolecular compounds have a sol-gel transition temperature, and show the sol state reversibly at temperatures lower than said transition temperature. Further, the wound dressing of the present invention contains macromolecular compounds that comprise multiple blocks with a cloud point and hydrophilic blocks that are bonded; have a sol-gel transition temperature in an aqueous solution; and show a liquid state (sol state) reversibly at temperatures lower than said sol-gel transition temperature. In the present invention, further, said sol-gel transition temperature is preferably higher than 0° C. but not higher than 40° C.

Further, the present invention provides a composition for a wound dressing that contains macromolecular compounds and antibacterial agents, wherein said macromolecular compounds comprise multiple blocks with a cloud point and hydrophilic blocks that are bonded; have a sol-gel transition temperature in an aqueous solution; and show a liquid state (sol state) reversibly at temperatures lower than said sol-gel transition temperature.

Further, the present invention provides a composition for a wound dressing that contains macromolecular compounds and a wound healing promoter, wherein said macromolecular compounds comprise multiple blocks with a cloud point and hydrophilic blocks that are bonded; have a sol-gel transition temperature in an aqueous solution; and show a liquid state (sol state) reversibly at temperatures lower than said sol-gel transition temperature.

In terms of its shape and structure, the present invention provides a wound dressing containing macromolecular compounds and formed into the shape of a film, woven cloth, non-woven cloth, sponge or gel-film, wherein said macromolecular compounds comprise multiple blocks with a cloud point and hydrophilic blocks that are bonded; have a sol-gel transition temperature in an aqueous solution; and show a liquid state (sol state) reversibly at temperatures lower than said sol-gel transition temperature.

The present invention further provides a wound dressing consisting of a supporting member comprising a film, woven cloth, non-woven cloth or sponge which is coated with macromolecular compounds, wherein said macromolecular compounds comprise multiple blocks with a cloud point and hydrophilic blocks that are bonded; have a sol-gel transition temperature in an aqueous solution; and show a liquid state (sol state) reversibly at temperatures lower than said sol-gel transition temperature.

### BRIEF DESCRIPTION OF THE DRAWINGS

FIG. 1 is a microscopic photograph of an uncoated gauze (magnification 32).

FIG. 2 is a microscopic photograph of a TGP-coated gauze (a wound dressing of the present invention) to be specifically described later (magnification 32).

FIG. 3 is a scanning electron-microscopic photograph of a TGP-coated sponge (a wound dressing of the present invention) (magnification 70 for FIG. 3(a) and 560 for FIG. 3 (b))

FIG. 4 is a scanning electron-microscopic photograph of an uncoated gauze after animal experiments (magnification 84).

FIG. 5 in a scanning electron-microscopic photograph of a wound dressing of the present invention (TGP coated gauze) after animal experiments (magnification 84)

FIG. 6 is an HE (hematoxylin-eosin) dyed image of a specimen of an uncoated gauze after animal experiments (magnification 1000)

FIG. 7 is an HE-dyed image of a specimen of a wound dressing of the present invention (TGP coated gauze) after animal experiments (magnification 1000)

### DETAILED DESCRIPTION OF THE INVENTION

The present invention is described in more detail below. The hydrophilic portion of the above macromolecular compounds comprising the wound dressing of the present invention is necessary in order that said macromolecular compounds become water-soluble at temperatures lower than the above sol-gel transition temperature. The hydrophobic portion is necessary for said macromolecules to change into the gel state at temperatures higher than the sol-gel transition temperature. In other words, the bond between the hydrophobic portions is necessary for the formation of cross-links of gels.

The carrier used in the present invention utilizes the following properties of the bond between the hydrophobic portions in the above-mentioned macromolecular compounds or the hydrophobic bond.

Hydrophobic bonding force increases with increasing temperature and thus the strength and density of cross links increase with the temperature. For this reason, the wound dressing of the present invention keeps its gel state and maintains its function as wound dressing at temperatures higher than the sol-gel transition temperature. In the present invention, further, the sol-gel transition occurs reversibly because temperature dependency of the above hydrophobic bonding force is reversible.

The hydrophilic portions of the above-mentioned macromolecular compounds is necessary, as above stated, in order that the macromolecular compounds change into water soluble substances at temperatures lower than the sol-gel transition temperature so that said wound dressing will be easily removed from the wound when replacing it or the wound is healed, The hydrophilic portions are also necessary to prevent said macromolecular compounds from aggregating and precipitating, and keep the water-containing gel state at temperatures above the sol-gel transition temperature. The above aggregation and precipitation may occur when the hydrophobia bonding force is increased too much at temperatures above said sol-gel transition temperature.

Examples of macromolecular compounds having a sol-gel transition temperature in aqueous solutions and showing a sol state reversibly at temperatures lower than said sol-gel transition temperature include, for example, polyalkylene oxide block copolymers represented by block copolymers of polypropylene oxides and polyethylene oxides, etherificated cellulose such as methyl cellulose and hydroxypropyl cellulose, and chitosan derivatives (K. R. Holme et al, Macromoleculars, 24, 38266, 1991).

A wound dressing gel using Pluronic F-127 (BASF Wyandotte Chemical Co.) comprising polypropylene oxides with polyethylene oxides bonded to both ends thereof has been developed (R. M. Nalbandian et al, J. Biomed. Mater. Res., 6, 533, 1972; J. Biomed. Mater. Res., 12, 1135, 1987).

An aqueous solution of Pluronic F-127 of a high concentration is known to be a hydrogel at temperatures above about 20° C. and returns to an aqueous solution at lower temperatures. The gel state, however, is maintained only for high concentrations above about 20 wt %. Further, even if the material is maintained at a gelation temperature and at a high concentration of above about 20 wt %, the gels are dissolved if water in added. If the Pluronic F-121 gels are applied to the wound, therefore, they are dissolved by the exudate from the wound and a stable gel state is hard to be maintained on the wound. Further, Pluronic F-127 with a relatively small molecular weight shows an extremely high osmotic pressure when it is in the gel state at high concentrations above about 20 wt %. Thus Pluronic F-127 easily permeates through cell membranes, and may adversely affect the wound.

For etherificated cellulose represented by methyl cellulose and hydroxylpropyl cellulose, on the other hand, the sol-gel transition temperature in as high as about 45° C. or above (M. Sarkar, J. Appl. Polym. Science, 24, 1073, 1979). If these etherificated celluloses are simply used as materials for the wound dressing of the present invention, these celluloses will take the sol state and are thus dissolved by exudate from the wound because the temperature at the wound is at least 37° C. or below.

As mentioned above, the problems of conventional macromolecular compounds having a sol-gel transition point in aqueous solutions and showing a sol state reversibly at temperatures lower than said transition temperature are:

1) the materials may be gelated at a temperature above the sol-gel transition temperature, but the gels are dissolved when water is added.

2) The sol-gel transition temperature is higher than the body temperature (37° C. or thereabout), and thus the materials are in the sol state at the body temperature,.

3) For effective gelation, the concentration of the macromolecular compounds in the aqueous solution must be increased considerably.

According to the study of the inventor of the present invention, the above problems are solved when the wound dressing is constructed by macromolecular compounds which comprise multiple blocks having a cloud point and hydrophilic blocks that are bonded together; have a sol-gel transition temperature in aqueous solutions; and show the sol state reversibly at temperatures lower than the sol-gel transition ion temperature.

The present invention is described in more detail below.

(Sol-Gel Transition Temperature)

The sol state, gel state and sol-gel transition temperature the defined below in the present invention. These definitions are found in a literature (Polymer Journal, 18 (5), 411-415, 1986).

One milliliter macromolecular solution is put in a test tube of 1 cm in inner diameter and allowed to rest in a water bath of a certain specified temperature for 12 hours. The test tube is then turned upside down vertically. When the interface (meniscus) between aqueous solution and air is changed by the weight of the aqueous solution (including flow-out of the aqueous solution), the macromolecular solution is defined to be in the sol state at the above specified temperature. If the above-mentioned interface (meniscus) between the aqueous solution and air does not change despite the weight of the aqueous solution when said test tube is turned upside down vertically, said aqueous solution is defined to be in the gel state at the above specified temperatures.

Using a macromolecular solution of, for example, about 3 wt % concentration in the above measurement, the above-mentioned specified temperature is increased slowly, say 1° C. at a time, to find the temperature at which the sol state turns into the gel state. This temperature is defined the sol-gel transition temperature, Alternatively, the above-mentioned specified temperature may be decreased gradually, say 1° C. at a time, to find the temperature at which the gel state changes into the sol state to determine the sol-gel transition temperature.

In the present invention, the sol-gel transition temperature should be higher than 0° C. but not higher than 50° C., preferably 4° C. or above but not higher then 37° C., from the viewpoint of preventing heat injury of the tissues in the wound. Macromolecular compounds with an adequate sol-gel transition temperature can be easily selected from chemical compounds which are named later in this document by using the above-mentioned screening method (sol-gel transition measuring method).

For the wound dressing of the present invention, the above-mentioned sol-gel transition temperature (a° C.) is preferably set between the temperature of the wound dressing in use (b° C.) and the temperature of said wound dressing when it is to be removed from the wound (c° C.). This means that the above three temperatures a, b, and c° C. have the relation b>a>c. More particularly, (b-a) should be from 1 to 35° C., preferably from 2 to 30° C., and (a-c) from 1 to 35° C., preferably from 2 to 30° C.

(Multiple Blocks Having a Cloud Point)

The blocks having a cloud point should be a macromolecular compound with a negative solubility temperature coefficient against water. More particularly, preferable macromolecular compounds include polypropylene oxide, copolymers of propylene oxide with other alkylen oxide, copolymers of poly N-substituted acrylamide derivatives with N-substituted methacrylamide derivatives, polyvinylmethylether, and polyvinylalcohol partial acetate. It is preferable for the above macromolecular compounds (blocks having a cloud point) to have a cloud point higher than 0° C. but not higher than 40° C. in order that for the macromolecular compounds of the present invention (compounds comprising multiple blocks with a cloud point and hydrophilic blocks that are bonded together) to have the sol-gel transition temperature between 0° C. and 40° C.

The cloud point is measured in the method described below, for example. About 1 wt % of aqueous solution of the above-mentioned macromolecular compounds (blocks with a cloud point) is cooled to make a uniform and transparent aqueous solution, which is then gradually heated at a rate of about 1° C./minute until said aqueous solution becomes cloudy for the first time, which is defined as the cloud point.

The following three examples of poly N-substituted acrylamide derivatives and poly N-substituted methacrylamide derivatives that may be used in the present invention:

Poly-X-acryloylpiperidine;

Poly-N-n-propylmethacrylamide;

Poly-N-isopropylacrylamide;

Poly-N, N-diethylacrylamide;

Poly-N-isopropylmethacrylamide;

Poly-N-cyclopropylacrylamide;

Poly-N-acryloyl pyrrolidine;

Poly-N,N-ethylmethylacrylamide;

Poly-N-cyclopropylmethacrylamide;

Poly-N-ethylacrylamide;

These macromolecules may be a homo polymer or a copolymer of a monomer comprising one of the above polymers and other monomers. The other monomers that comprise such a copolymer may be any of the hydrophilic or hydrophobic monomers. Generally, the cloud point increases when hydrophilic monomers are used for copolymerization, and decreases when hydrophobic monomers are used. It is thus possible to get macromolecular compounds with a desired cloud point (for example, higher than 0° C. but not higher than 40° C.) by selecting a monomer to be used for copolymerization.

The above-mentioned hydrophilic monomers include, but not limited to, N-vinylpirrolydone, vinylpyridine, acrylamide, methacrylamide, N-methylacrylamide, hydroxyethylmethacrylate, hydroxyethylacrylate, hydroxymethylmethacrylate, hydroxymethylacrylate, acrylic acid with acid group, methacrylic acid and its salts, vinylsulfonic acid, and styrenesulfonic acid, as well as N,N-dimethylanimoethylmethacrylate, N,N-diethylanimoethylmethacrylate, N,N-dimethylanimopropylacrylamino and their salts.

The above-mentioned hydrophobic monomers include, but not limited to, acrylate and methacrylate derivatives such as ethylacrylate, methylmetharylate, and n-butyl methacrylate, N-substituted alkylmethacrylamide derivatives such as N-n-butylmethacrylamide, vinyl, chloride, acrylonitrile, styrene, and vinyl acetate.

(Hydrophilic Blocks)

Hydrophilic blocks to bond the above-mentioned blocks having a cloud point include methyl cellulose, dextran polyethylene oxide, polyvinylalcohol, poly N-vinylpyrrolydone, polyvinylpyridine, polyacrylamide, polymethacrylamide, poly N-methylacrylamide, polyhydroxymethylacrylate, polyacrylic acid, polymethacrylic acid, polyvinylsulfonic acid, polystyrenesulfonic acid, and their salts; poly N,N-dimethylaminoethylmethacrylate, poly N,N-diethylaminoethylmethacrylate, poly N,N-dimethylaminopropylacrylamide and their salts.

The method to bond blocks having a cloud point with the above-mentioned hydrophilic blocks is not limited, and include a method to introduce a polymerizing functional group (acryloil group, for example) into one of the above-mentioned blocks and copolymerize it with a monomer that gives the other block.

The substance comprising blocks having a cloud point and the above hydrophilic blocks that are bonded together can also be derived by block copolymerization of monomers giving blocks having a cloud point with monomers giving hydrophilic blocks.

The bond between blocks having a cloud point and hydrophilic blocks can also be accomplished by chemical reaction between both blocks by introducing beforehand a reactive functional group (hydroxyl group, amino group, carboxyl group, and isocyanate group, for example) to each of them.

In this instance, generally, multiple reactive functional groups are introduced to the hydrophilic blocks.

The bond between polypropylene oxides having a cloud point and hydrophilic blocks can also be accomplished by repeatedly and sequentially polymerizing propylene oxides with monomers comprising “other water-soluble macromolecules” (such as ethylene oxides) using, for example, anion or cation polymerization. This produces block copolymers comprising polypropylene oxides and “other water-soluble macromolecules” (polyethylene oxides, for example) which are bonded. These block copolymers can also be derived by introducing polymerizing groups (such as acryloil group) into the ends of polypropylene oxides and then copolymerizing monomers comprising water-soluble macromolecules.

The macromolecular compounds used in the present invention can be also obtained by introducing a functional group which can be bonded by reaction to the functional group (a hydroxyl group, for example) located at the end of polypropylene oxides and allowing both functional groups to react to each other in the water-soluble macromolecules. Further, macromolecular compounds used in the present invention can be derived by connecting a material such as Pluronic F-127 (product name, made by BASF Co.,) which contains polyethylene glycol bonded to both ends of polypropylene glycol.

The macromolecular compounds of the present invention are perfectly dissolved in water in the sol state at temperatures below the cloud point because the above-mentioned “blocks having a cloud point” are water-soluble as are the hydrophilic blocks at said temperatures.

When the aqueous solution containing these macromolecular compounds is heated above the above-mentioned cloud point, the “blocks having a cloud point” present in the molecules become hydrophobic, and will associate between different molecules owing to the mutual hydrophobic action.

The hydrophobic blocks, on the other hand, remain water-soluble at this time (when the solution is heated above the cloud point), and thus the macromolecular compounds of the present invention generate a hydrogel of a three-dimensional network structure wherein the hydrophobic association areas between the blocks having a cloud point serve as cross-links. When this hydrogel is cooled below the cloud point of the “blocks having a cloud point” present in the molecules, said blocks become water-soluble; cross-links of hydrophobic association are released; the hydrogel structure disappears; and the macromolecular compounds of the present invention become a perfect aqueous solution again. Sol-gel transition of the macromolecular compounds of the present invention thus has a perfect reversibility because it is based on the reversible hydrophilic and hydrophobic change at the cloud point of blocks present in the molecules.

According to the knowledge of the inventor of the present invention, once the macromolecular compounds used in the present invention are gelated at a temperature above the sol-gel transition temperature, the gel will not be dissolved even if a large quantity of water (about 0.1 to 100 times the gel by volume) is added.

The wound dressing of the present invention contains at least the above-mentioned macromolecular compounds having a sol-gel transition temperature, but may contain other components as required, The “other components” include, in addition to antibacterial agents and wound healing promoters described later, anodynes, etc., for example. In this case, the “other components” should be from 0.5 to 30 parts, preferably from 1 to 10 parts per 100 (weight) parts of macromolecular compounds having a sol-gel transition temperature.

(Antibacterial Agents)

The external-use antibacterial agents extensively used in clinical examination and diagnosis in recent years are preferably used as the antibacterial agents in the present invention. More specifically, for example, silver nitrate, para-aminobenzenesulfamide, gentamycin, silver sulfadiazine, nalidixcic acid, piromidic acid, pipemidic acid norfloxacin, ofloxain, and cyprofloxacin are used, but these are not the only antibacterial agents used in the present invention. The above antibacterial agents are used in a quantity of from 0.5 to 30 parts, preferably from 1 to 10 parts, per 100 parts of macromolecular compounds of the present invention.

(Wound Healing Promoter)

Extracellular matrices with the effect of enhancing affinity to the tissues and promoting epitherial growth are particularly favorably added in the present invention as a substance to promote wound healing. More specifically, various types of collagen, fibronectin, vitronectin, laminin, proteoglycan, and glycosaminoglycan are used as extracellular matrices. Besides extracellular matrices, gelatins, a thermally denatured substance of collagen, etc., having the same effect, may be used in the same way as the above-mentioned extracellular matrices.

The above wound healing promoters are used in a quantity of from 0.1 to 50 parts, preferably from 1 to 20 parts, per 100 parts of macromolecular compounds of the present invention.

(Method to Manufacture Wound Dressing)

The method to manufacture wound dressing of the present invention is described in concrete below.

Wound dressings comprising a film of the above-mentioned macromolecular compounds can be produced by, for example, dissolving the macromolecular compounds of the present invention in an organic solvent, or by dissolving in water at a temperature lower than the sol-gel transition temperature, and then selecting solvent casting for said solution. Another method is to coat a supporting member made of woven cloth, nonwoven cloth, sponge or film represented by gauze with said macromolecular solution and then remove the solvent by drying. This particular method produces wound dressings comprising a supporting member on the surface of which said macromolecular compounds are coated. For this type of wound dressings having a supporting member on the surface of which the above-mentioned macromolecular compounds are coated, the quantity of the above-mentioned macromolecular compounds to be coated should be from 0.1 to 50 mg per 1 cm2 of supporting member (by dry weight), preferably from 0.5 to 10 mg per 1 cm2 of supporting member (by dry weight).

Further, the above-mentioned antibacterial agents or healing promoters may be mixed in the above-mentioned macromolecular solution, followed by solvent casting which is performed in the same manner as above. This particular method produces macromolecular compound film containing the above materials, or wound dressings comprising a supporting member on the surface of which said macromolecular compound film is coated.

It is possible to manufacture sponges of the above-mentioned macromolecular compounds having interconnected porosity by using the known so-called freeze-dry method. In this method, the above-mentioned macromolecular compounds are dissolved in water at a temperature lower than the sol-gel transition temperature; the aqueous solution of said macromolecular compounds is cooled to below the freezing point of water; and the resulting frozen substances are vacuum dried. It is also possible to manufacture wound dressings comprising a supporting member whose surface is coated with macromolecular compound sponges with interconnected porosity. In this particular method, an aqueous solution of the above-mentioned macromolecular compounds is coated on a woven cloth, non-woven cloth, sponge or film-like supporting member represented by a gauze at a temperature lower than the sol-gel transition temperature and said coated material is freeze-dried in the same manner as described above.

Further, the above-mentioned macromolecular compounds are dissolved in water at a temperature lower than the sol-gel transition temperature, and the above-mentioned antibacterial agents or healing promoters may be dissolved or mixed in said macromolecular solution, followed by the freeze-dry process, which is performed in the same manner as above. This particular method produces a macromolecular compound interconnected porous sponge containing the above substances, or wound dressings comprising a supporting member on the surface of which said macromolecular compound inter-connected porous sponge is coated.

(How to use the Wound Dressing)

An example of how to use of the wound dressing of the present invention is described below in concrete.

The wound dressing of the present invention is attached to a wound to cover it while the wound dressing is dry or after immersing it in a physiological saline solution of a temperature higher than the sol-gal transition temperature of said macromolecular compounds.

When replacing said wound dressing or removing it from the wound after the wound has been healed, said wound dressing is wetted by water or physiological saline solution of a temperature below said sol-gel transition temperature (preferably about 4° C. or thereabout) in order to change the macromolecular compounds present in said wound dressing into water-soluble substance, The wound dressing can then be peeled off from the wound. The base material make of the macromolecular compounds of the wound dressing itself, or the macromolecular compounds coated on, the supporting member of the wound dressing in contact with the wound is dissolved, with the result that the wound dressing is removed extremely easily. For this reason, the wound dressing can be replaced or peeled off without damaging the wound in the process of healing and without giving pain to the patient.

Working examples are shown below to further describe the present invention in detail. The scope of the present invention is limited by the scope of claim, and is not limited by these working examples.

### WORKING EXAMPLE 1

160 mol ethylene oxide was added to 1 mol trimethylol propane by cation polymerization to derive polyethylene oxide triol, 0.02 mol polyethylene oxide trial was dissolved in 1000 ml distilled water; 0.1 mol potassium permanganate was added; and oxidation was performed for 60 minutes at 25° C. to derive tricarboxyl-terminated polyethylene oxide.

10 g tricarboxyl-terminated polyethylene oxide and 10 g diamino-terminated polypropylene oxide (average degree of polymerization of propylene oxide was about 65, made by Texaco Chemical Co., U.S.: Jeffamine D-4000) were dissolved in 1000 ml carbon tetrachloride; 1.2 g N,N′-carbonyldimidazole was added; and the solution was allowed to react for 6 hours under the boiling point reflux. The resultant liquid was cooled, filtered, and the solvents were removed under reduced pressure. The residues were vacuum dried to derive macromolecular compounds (TGP) of the present invention.

The above macromolecular compounds were dissolved in distilled water to a concentration of 8 wt %. When this aqueous solution was slowly heated, the viscosity increased gradually starting at 5° C. Solidification started at about 10° C. to form hydrogels. The hydrogels returned to aqueous solutions when they were cooled to 5° C. this change was observed reversibly and repeatedly. A large quantity of distilled water was input into the hydrogel at 25° C., but the hydrogel did not dissolve.

The TGP obtained in the above method was dissolved in acetone to derive acetone solution of 5 w/w % concentration. A gauze to the Japanese Pharmacopoeia (type I) was immersed in said acetone solution to be fully wetted in said solution. The gauze was then removed from solution; nitrogen gas was sprayed to the gauze after filtering said was by a Millex-FG filter (product name, made by Millipore) to remove extra solution from the gauze. The gauze was dried for 10 minutes at the room temperature. The TGP was coated on the gauze by repeating the above procedures three times. The weight of the gauze was measured before and after the coating. According to the result of the measurement, the dry weight of the coated TGP was approximately 5 mg per 1 cm2 of gauze.

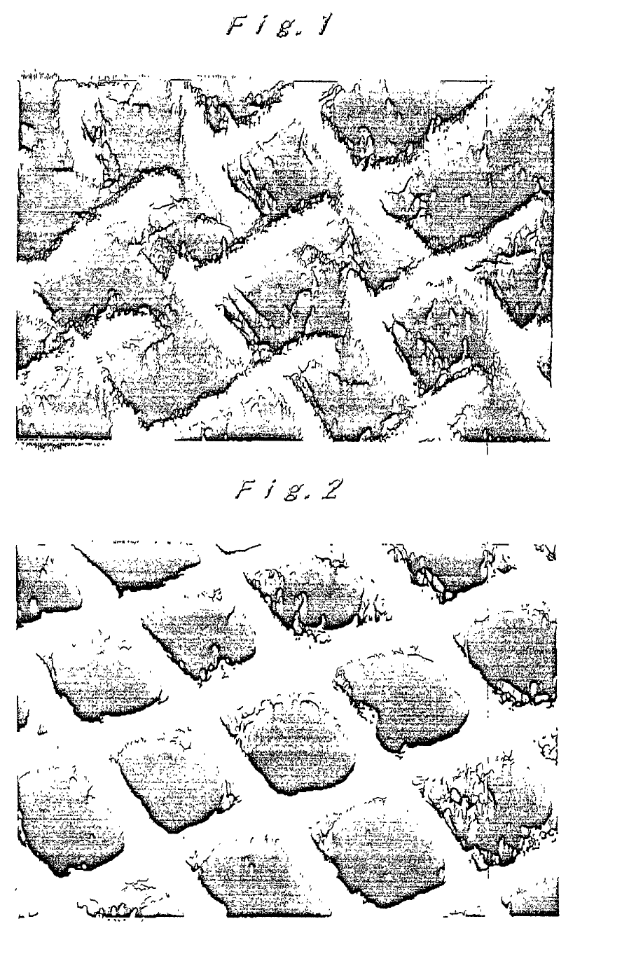

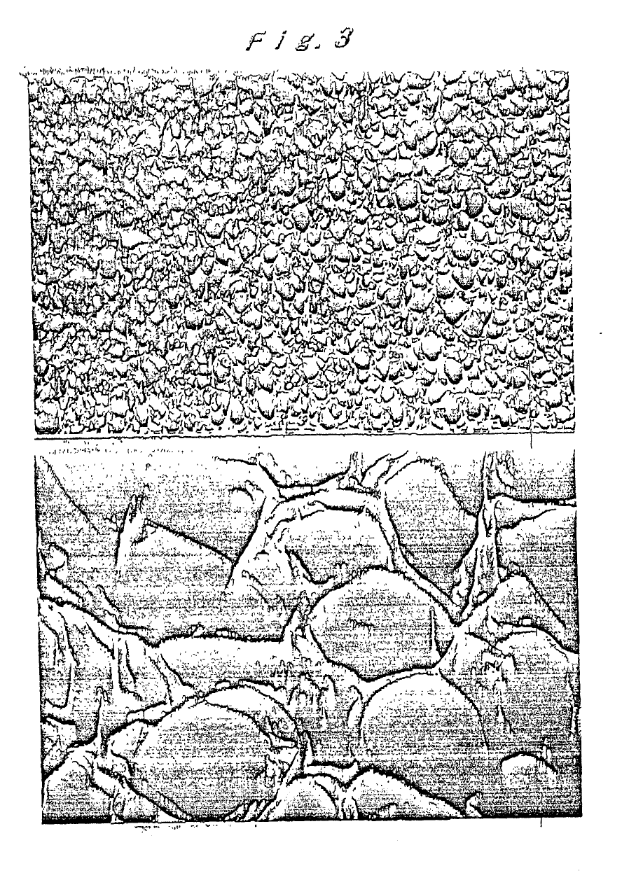

Microscopic photographs of the gauze before and after the coating are shown in FIGS. 1 and 2, respectively (magnification 32 for both). The gauze structure shows no significant change before and after the coating including the diameter of holes of the gauze, except that the disturbed fine fibers observed before the coating disappeared after the coating.

As a comparative study, the following experiments were carried out to simulate the elution of the TGP in TGP coated gauze for the cases when the gauze is in use and when it is wetted with water of a temperature lower than the sol-gel transition temperature for removal of the wound dressing from the wound.

100 cm2 TGP coated gauze was immersed in 30 ml water at 37° C. for one week. After one week, the quantity of eluted TGP in the water was measured by the evaporation-to-drying method. The measurement shows that 0.125 mg TGP per 1 cm2 of gauze (approximately 2.5% of the coated TGP) has eluted. Next, the temperature of the above solution with the gauze in it was lowered by approximately 4° C. and the solution was allowed to rest for 5 minutes. The amount of eluted TGP from said gauze was measured by the evaporation-to-drying method. The measurement shows that 3.84 mg TGP per 1 cm2 of gauze (approximately 77% of the coated TGP) was eluted.

The above result of experiments suggests that the wound dressing of the present invention keeps the function of a wound dressing without dissolving into the exudate secreted from the wound or into aqueous solution of various medicines that may be applied from outside to the wound dressing. The result of the experiments further suggests that, when removing the wound dressing of the present invention from the wound, the TGP comprising said wound dressing will be dissolved to wake removal of the wound dressing easy when the temperature of said wound dressing is decreased to below the sol-gel transition temperature and the wound dressing is wetted by water of a temperature lower than the sol-gel transition temperature that is sprayed from outside the wound dressing.

### WORKING EXAMPLE 2

The TGP used in working example 1 was dissolved in distilled water at 4° C. to derive 1 w/w % aqueous solution. A gauze (the Japanese Pharmacopoeia (type I)) was placed fully extended at the bottom of a plastic dish of 9.5 cm in diameter, and 15 ml TGP aqueous solution was powred into the dish to fully soak the gauze.

The above aqueous solution was thoroughly frozen in a refrigerator at −80° C. for 2 hours. The frozen solution was then freeze-dried in a vacuum dryer over one day to produce wound dressing of the present invention. Scanning electron microscopic photographs (magnification 70 and 560) of the surface of said wound dressing are shown in FIGS. 3 (a) and 3(b), respectively. The wound dressing used in the experiment was a sponge-like dressing with interconnected holes of approximately 30 um in diameter.

### WORKING EXAMPLE 3

The TGP used in working example 1 was dissolved in acetone to derive acetone solution of 5 w/w % concentration. Further, silver sulfadiazine (Tanabe Seiyaku) was mixed in said acetone solution as antibacterial agent to derive a solution of approximately 0.2 w/w %. A gauze coated with the antibacterial agent-containing TGP was manufactured in exactly the same method as used in working example 1 except that the above said solution was used. The content of the antibacterial agent was approximately 4% with respect to the coated TGP or 0.08 mg per 1 cm2 of the wound dressing.

The wound dressing produced in the above method was observed under a scanning electron microscope in the same way as in working example 1. The structure was completely the same as that of working example 1.

The antibacterial property of the above antibacterial agent-containing wound dressing was tested as follows.

20 ml NAC agar culture media (made by Eiken Kagaku K.K) were placed in a plastic dish of 90 mm in diameter. P. aeruginose, (GN11189) was seeded on the prepared agar culture media at a concentration of 1×10 cells/cm2. The antibacterial agent-containing wound dressing (3×3 cm) obtained in the above-mentioned process was placed on said agar culture media for cultivation in a 37° C. incubator for 2 days. After cultivation, the agar under said wound dressing was partly cut to obtain a cube measuring 1×1×0.3 cm. The cube was homogenized in 10 ml sterilized physiological saline solution to derive test solution A.

As control experiments, a part of the agar which was not covered by the above-mentioned wound dressing was cut to obtain a cube measuring 1×1×0.3 cm to prepare test solution B in the same manner as for test solution A.

0.1 ml test solution A, and 0.1 ml test solution B which was diluted by 100,000 times, were seeded on a newly prepared agar culture medium of the same specifications as above, respectively. The test solutions were cultured for one day at 37° C. and the number of bacteria was estimated from the number of the colonies formed. The concentration of P. aeruginosa in test solution B was approximately 4×10⁷/ml while no P. aeruginosa was detected in test solution A. This experiment indicates that the antibaterial effect of the antibacterial agent-containing wound dressing of the present invention is extremely good.

### WORKING EXAMPLE 4

The TGP used in working example 1 was dissolved in distilled water of 4° C. to prepare 1 w/w % aqueous solution. The pH of said TGP aqueous solution was adjusted to 3, and 4 ml 0.5 w/v % calf pepsin soluble type I collagen solution (KOKEN CELLLGEN I-PC, made by Koken K.K.) was added to 20 ml TGP aqueous solution (pH adjusted to 3) at 4° C. to prepare an aqueous solution containing 1 part of collagen per 10 parts of the TGP.

A gauze (the Japanese Pharmacopoeia (type I)) was placed fully extended at the bottom of a plastic dish of 9.5 cm in diameter, and 15 ml of the above-mentioned aqueous solution was put in the dish to immerse the gauze. The above aqueous solution was thoroughly frozen in a refrigerator at −80° C. for 2 hours. The frozen solution was then freeze-dried in a vacuum dryer over one dry to produce the wound dressing of the prevent invention. The surface of said wound dressing was observed under a scanning electron microscope to find that the sponge structure was the same as one observed in working example 2.

### WORKING EXAMPLE 5

Fight-weak-old Wister rats(male) were shaved over en area ranging from the left back to the latoroabdominal region under ether anesthesia followed by antisepsis to make a 4×4 cm of a full-thickness skin wound. The surface was covered by the TGP-wated gauze made in working example 1, further covered by 8 sheets of sterilized plain gauze, followed by pressure fixing using elastic bandage for one week.

For controls, a full thickness skin wound was prepared by the same procedure and the wound surface was covered with the sterilized unwated gauze (the Japanese Pharmacopoeia (type I)) used in working example 1.

After one week of covering, a sufficient amount of woled saline solution at 4° C. was poured on the gauze to solulrilize the TGP after removing the elastic bandage under ether anesthesia and the gauze was slowly lifted by picking up both sides of the gauze. For controls, the gauzes were also removed by the same procedure. The first 5 gauzes from the top were removed easily but the remaining 3 gauses adhered to the wound surfaces and it was difficult to remove. When these 3 gauses were removed by force, a large quantity of bleeding from the surface was seen. On the other hand, the removal of the TGP-wated gauze from the wound surface could be achieved easily and bleeding from the wound surface was not seen.

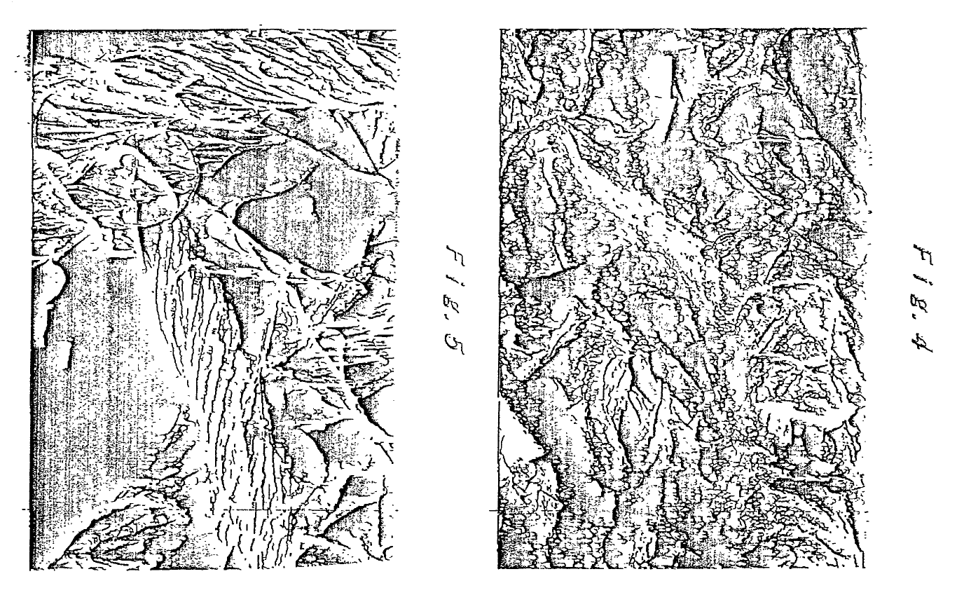

The gauzes and the wound dressing removed from the wound were examined by a scanning electron microscope. The result is shown in FIGS. 4 and 5, respectively (magnification 84 for both). The arrows in the figures show the adhesion of tissue fragments. As seen in these figures, a large amount of granulation tissues were attached to the textures of the gauze (control experiment) (FIG. 4) while only a very small amount of granulation tissues were attached to the textures of the wound dressing of the present invention (FIG. 5).

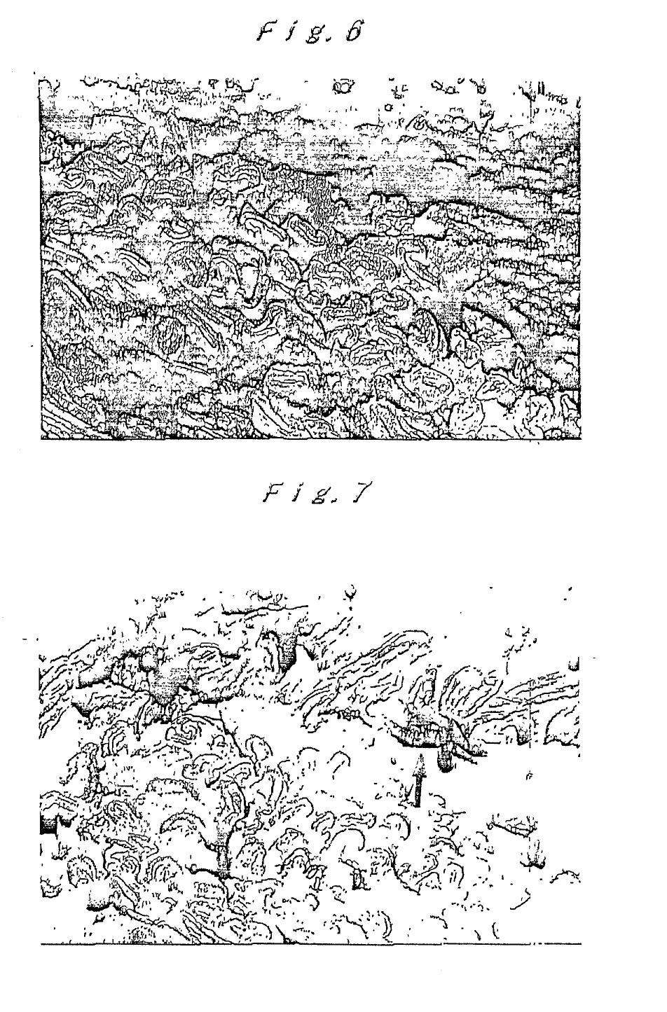

Further, the gauzes and the wound dressing removed from the wound were fixed in a 10% formalin physiological saline solution and stained with HE (hematoxylin-eosin) for the microscopic examination. FIGS. 6 and 7 (magnification 1000 for both) show the microscopic photographs of specimens of said uncoated gauzes and wound dressing, of the present invention, respectively. In FIGS. 6 and 7, the arrows indicate the tissues fragments attached to the gauze or the wound dressing.

As these photographs show, a large amount of granulation tissues adhered attached to the gauze (control experiment, FIG. 6) while very little adhesion of the tissues fragments was observed on the wound dressing of the present invention (FIG. 7).

### WORKING EXAMPLE 6

The sponge-like wound dressing of the present invention prepared in working example 2 was placed on the full-thickness skin wound in a rat in exactly the same manner as described in working example 5 for one week. After the one-week covering, the test specimen was peeled off from the wound in the same manner as in working example 5.

It is found that when using the wound dressing of the present working example, the peelability from the wound is further more improved than wound dressing used in working example 5. Microscopic findings of the removed wound dressing and the observations of the fixed sample specimens were approximately the same as those in working example 5. In other words, adhesion of the granulation tissues fragments to the above wound dressing was scarcely observed.

### WORKING EXAMPLE 7

Animal experiments were carried out using the gauze wated with TRP containing silver sulfadiazine prepared in working example 3 in exactly the same manner as in working example 5.

Peelability of said wound dressing from the wound, adhesion of granulation tissues fragments to the removed wound dressing, and residues of the wound dressing in the wound tissues were exactly the same as for the wound dressing of the present invention prepared in working example 5. That is, the presence of antibacterial agents did not affect the performance of the wound dressing of the present invention.

### WORKING EXAMPLE 8

Animal experiments were carried out for collagen-containing wound dressing of the present invention prepared in working example 4 in exactly the same manner as in working example 5.

Peelability of said wound dressing from the wound, adhesion of granulation tissues fragments to the removed wound dressing, and residues of the wound dressing in the wound tissues were exactly the same as for the wound dressing of the present invention prepared in working example 6. That is, the presence of collagen did not affect the performance of the wound dressing of the present invention. The healing condition of the wound after removing said wound dressing was observed histologically to find that the collagen-containing wound dressing is better than the wound dressing of the present invention that does not contain collagen.

The present invention as described above in detail thus provides a wound dressing comprising macromolecular compounds, wherein said macromolecular compounds contain multiple blocks having a cloud point and hydrophilic blocks, and have a sol-gel transition temperature. The wound dressing of the present invention has excellent absorption and discharge capability for exudate secreted from the wound, and excellent peelability from the wound, both of which are based on the thermally reversible gelation property of the macromolecular compounds having a sol-gel transition temperature.

When the wound dressing of the present invention contains, in particular, macromolecular compounds whose sol-gel transition temperature is between body temperature and room temperature, said wound dressing, when applied to a wound, will not be dissolved by exudate secreted from the wound nor by aqueous solutions containing various medicines applied external of the wound dressing, and maintain the function of a wound dressing. When replacing said wound dressing or removing it from the healed wound, water or physiological saline solution of a temperature lower than said sol-gel transition temperature (most preferably 4° C. or thereabout in practical applications) is sprayed over said wound dressing in order to bring down the temperature of said wound dressing to below said sol-gel transition temperature and make it wetted so that said macromolecular compounds are turned to soluble. As a result, said wound dressing can be removed from the wound easily.

When the wound dressing of the present invention is used, unlike conventional wound dressings, virtually no damage is given to the wound when removing it. In addition, the time required for changing the wound dressing and the accompanying pain to the patient are decreased drastically.

We claim:

1. A wound dressing containing macromolecular compounds, wherein said macromolecular compounds comprise multiple blocks having a cloud point and hydrophilic blocks, have a sol-gel transition temperature in aqueous solutions, and indicate a liquid state reversibly at temperatures lower than said sol-gel transition temperature.

2. A wound dressing claimed in claim 1 wherein said sol-gel transition temperature is higher than 0° C. but not higher than 40° C.

3. A wound dressing claimed in claim 1 wherein said wound dressing contains antibacterial agents.

4. A wound dressing claimed in claim 1 wherein said wound dressing contains wound healing promoters.

5. A wound dressing claimed in claim 1 wherein the shape of said macromolecular compounds is film-, woven cloth-, non-woven cloth-, sponge- or gel film-type.

6. A wound dressing claimed in claim 1 wherein said macromolecular compounds are coated on the surface of a supporting member.

7. A wound dressing claimed in claim 6 wherein the shape of said supporting member is film-, woven cloth-, non-woven cloth-, or sponge-type.
